(19) United States
(12) Patent Application Publication
(10) Pub. No.: US 2005/0033393 A1
(43) Pub. Date: Feb. 10, 2005

(54) Apparatus and method for implanting an electrical stimulation system and a paddle style electrical stimulation lead

(75) Inventor: Terry Daglow, Allen, TX (US)

Correspondence Address: BAKER BOTTS L.L.P., 2001 ROSS AVENUE, 6TH FLOOR, DALLAS, TX, 75201-2980, US

(73) Assignee: Advanced Neuromodulation Systems, Inc.

(21) Appl. No.: 10/637,342

(22) Filed: Aug. 8, 2003

(51) Int. Cl. A61N 1/05

(52) U.S. Cl. 607/116; 604/171

(57) ABSTRACT

In one embodiment, an introducer is provided for implanting an electrical stimulation lead to enable electrical stimulation of nerve tissue. The introducer includes an outer sheath and an inner penetrator. The outer sheath is configured to accommodate insertion of the electrical stimulation lead through the outer sheath and may be inserted into a human body near the nerve tissue. The inner penetrator is removably housed within the outer sheath and includes an inner channel configured to accommodate a guide wire. The inner penetrator may be advanced along the guide wire to a desired location relative to the nerve tissue and removed from the outer sheath leaving the outer sheath substantially in position for insertion of the electrical stimulation lead through the outer sheath into position proximate the nerve tissue.

### TECHNICAL FIELD OF THE INVENTION

This invention relates generally to electrical stimulation leads for medical applications and in particular to an apparatus and method for implanting an electrical stimulation system that includes a paddle style electrical stimulation lead.

### BACKGROUND

Electrical energy is applied to the spinal cord and peripheral nerves to treat regions of the body that are affected by chronic pain from a variety of etiologies. One method of delivering electrical energy is to implant an electrode and position it in a precise location adjacent the spinal cord such that stimulation of the electrode causes a subjective sensation of numbness or tingling in the affected region of the body, known as “paresthesia.” Pain managing electrical energy is commonly delivered through electrodes positioned external to the dura layer surrounding the spinal cord. The electrodes may be carried by either of two primary vehicles: a percutaneous lead and a laminotomy or “paddle” lead.

Percutaneous leads commonly have three or more equally-spaced electrodes. They are positioned above the dura layer using a needle that is passed through the skin, between the desired vertebrae and onto the top of the dura. Percutaneous leads deliver energy radially in all directions because of the circumferential nature of the electrode. Percutaneous leads can be implanted using a minimally invasive technique. In a typical percutaneous lead placement, a trial stimulation procedure is performed to determine the optimal location for the lead. Here, a needle is placed through the skin and between the desired vertebrae. The percutaneous lead is then threaded through the needle into the desired location over the spinal cord dura. Percutaneous leads may also be positioned in other regions of the body near peripheral nerves for the same purpose.

Laminotomy or paddle style leads have a paddle-like configuration and typically possess multiple electrodes arranged in one or more independent columns. Paddle style leads provide a more focused energy delivery than percutaneous leads because electrodes may be present on only one surface of the lead. Paddle style leads may be desirable in certain situations because they provide more direct stimulation to a specific surface and require less energy to produce a desired effect. Because paddle style leads are larger than percutaneous leads, they have historically required surgical implantation through a procedure known as partial laminectomy that requires the resection and removal of vertebral tissue.

### SUMMARY OF THE INVENTION

The present invention provides an introducer and process for implanting a paddle style electrical stimulation lead.

In one embodiment, an introducer is provided for implanting an electrical stimulation lead to enable electrical stimulation of nerve tissue. The introducer includes an outer sheath and an inner penetrator. The outer sheath is configured to accommodate insertion of the electrical stimulation lead through the outer sheath and may be inserted into a human body near the nerve tissue. The inner penetrator is removably housed within the outer sheath and includes an inner channel configured to accommodate a guide wire. The inner penetrator may be advanced along the guide wire to a desired location relative to the nerve tissue and removed from the outer sheath leaving the outer sheath substantially in position for insertion of the electrical stimulation lead through the outer sheath into position proximate the nerve tissue.

In another embodiment, a method is provided for implanting an electrical stimulation lead to enable electrical stimulation of nerve tissue. The method includes inserting a needle into tissue, removing the needle, forming a tract for the electrical stimulation lead by spreading tissue using an introducer including an outer sheath and an inner penetrator removably housed within the outer sheath, removing the inner penetrator, leaving the outer sheath substantially in position, and inserting the electrical stimulation lead through the outer sheath until the electrical stimulation lead is positioned proximate the nerve tissue.

In another embodiment, a method is provided for implanting an electrical stimulation lead in a minimally invasive percutaneous manner to enable electrical stimulation of a human's spinal nerve tissue. The method includes inserting a needle into the human's epidural space and inserting a guide wire through the needle until an end of the guide wire is positioned in the epidural space at a desired location relative to the spinal nerve tissue to be stimulated. The position of the guide wire in the epidural space is verified using fluoroscopy. The needle is then removed, leaving the guide wire substantially in position. An introducer including an outer sheath and an inner penetrator, the inner penetrator removably housed within the outer sheath and including an inner channel configured to accommodate the guide wire, is then advanced along the guide wire until an end of the inner penetrator of the introducer is positioned in the epidural space at a desired location with respect to the spinal nerve tissue to be stimulated, the outer sheath of the introducer forming a tract as the inner penetrator of the introducer advances along the guide wire. The outer sheath of the introducer has a width of at least approximately two times its height. The position of the introducer in the epidural space is verified using fluoroscopy. The guide wire and the inner penetrator of the introducer are then removed, leaving the outer sheath of the introducer substantially in position. The paddle style electrical stimulation lead is then inserted through the outer sheath of the introducer until the paddle style electrical stimulation lead is positioned in the epidural space proximate the nerve tissue to be stimulated. The position of the paddle style electrical stimulation lead in the epidural space is verified using fluoroscopy.

In another embodiment, a method of implanting a system to enable electrical stimulation of a human's nerve tissue is provided. The method includes inserting a needle into tissue proximate the nerve tissue to be stimulated. A guide wire is inserted through the needle until an end of the guide wire is positioned at a desired location relative to nerve tissue to be stimulated. The needle is then removed leaving the guide wire substantially in position. An introducer including an outer sheath and an inner penetrator, the inner penetrator removably housed within the outer sheath and including an inner channel configured to accommodate the guide wire, is then advanced along the guide wire until an end of the inner penetrator of the introducer is positioned at a desired location relative to the peripheral nerve tissue to be stimulated, the outer sheath of the introducer forming an insertion tract as the inner penetrator of the introducer advances along the guide wire. The guide wire and the inner penetrator of the introducer are then removed, leaving the outer sheath of the introducer substantially in position. An electrical stimulation lead is then inserted through the outer sheath of the introducer until the electrical stimulation lead is positioned proximate the peripheral nerve tissue to be stimulated. The outer sheath is removed. The electrical stimulation lead is connected to a generator. A subcutaneous pocket is created for the generator and the generator is inserted into it.

In another embodiment, a system for implanting an electrical stimulation lead to enable electrical stimulation of a human's spinal nerve tissue is provided. The introducer includes an outer sheath and an inner penetrator. The outer sheath is configured to accommodate insertion of the electrical stimulation lead through the outer sheath and may be inserted through the human's skin and into the human's epidural space. The inner penetrator is removably housed within the outer sheath and includes an inner channel configured to accommodate a guide wire. The inner penetrator may be advanced along the guide wire until an end of the inner penetrator is positioned in the epidural space at a desired location relative to spinal nerve tissue to be stimulated, the outer sheath forming an insertion tract as the inner penetrator advances along the guide wire. The inner penetrator is configured to be removed from the outer sheath leaving the outer sheath substantially in position for insertion of the electrical stimulation lead through the outer sheath into position proximate the spinal nerve tissue to be stimulated. The system also includes an implantable generator to power the electrical stimulation lead.

In another embodiment, a needle for introduction of a paddle style electrical stimulation lead near a spinal column of a human includes in combination a body having a proximal end and a distal end, a lumen having a continuous oblong cross section defined by a solid outer wall, and a stylet having a handle at a proximal end and a solid body extending from the proximal end to a distal end and adapted to be inserted within the lumen, the improvement comprising a raised circumferential ridge configured to create resistance when the circumferential ridge contacts the human's ligamentum flavum.

Particular embodiments of the present invention may provide one or more technical advantages. For example, certain embodiments may allow a paddle style electrical stimulation lead to be inserted using a minimally invasive procedure, using an introducer, rather than a partial laminectomy or other more invasive surgical procedure. Certain embodiments may provide a guide wire, introducer and paddle style electrical stimulation lead composed in part or entirely of radio-opaque material to allow for fluoroscopic verification of the position of the guide wire, introducer and lead. Certain embodiments may provide an inner penetrator including a hollow tip configured to extend beyond the outer sheath, the tip having a raised circumferential ridge configured to create resistance when the circumferential ridge contacts the human's tissue. Certain embodiments may provide all, some, or none of these advantages. Certain embodiments may provide one or more other technical advantages, one or more of which may be readily apparent to those skilled in the art from the figures, description and claims included herein.

### BRIEF DESCRIPTION OF THE DRAWINGS

To provide a more complete understanding of the present invention and the features and advantages thereof, reference is made to the following description taken in conjunction with the accompanying drawings, in which:

FIG. 1A illustrates an example introducer for implanting a paddle style electrical stimulation lead;

FIG. 1B illustrates an example inner penetrator of an introducer for implanting a paddle style electrical stimulation lead;

FIG. 1C illustrates an example of an outer sheath of an introducer for implanting a paddle style electrical stimulation lead;

FIG. 1D illustrates an example of a tip of an introducer for implanting a paddle style electrical stimulation lead;

FIG. 1E illustrates an example of a tip of an outer sheath of an introducer for implanting a paddle style electrical stimulation lead;

FIG. 1F illustrates a side view of an example of a tip of an introducer for implanting a paddle style electrical stimulation lead;

FIG. 2A illustrates an example of a needle inserted into a human's epidural space;

FIG. 2B illustrates an example of a guide wire being inserted through a needle into a human's epidural space;

FIG. 2C illustrates an example of an introducer being inserted over a guide wire into a human's epidural space;

FIG. 2D illustrates an example of an inner penetrator being removed from the outer sheath of an introducer in a human's epidural space;

FIG. 2E illustrates an example of a paddle style lead being inserted through an introducer into a human's epidural space;

FIG. 2F illustrates an example of a paddle style lead implanted in a human's epidural space;

FIG. 3A illustrates an example of a stimulation system;

FIG. 3B illustrates an example of a stimulation system; and

FIG. 4 is a flow chart describing steps for implanting a stimulation system.

### DESCRIPTION OF EXAMPLE EMBODIMENTS

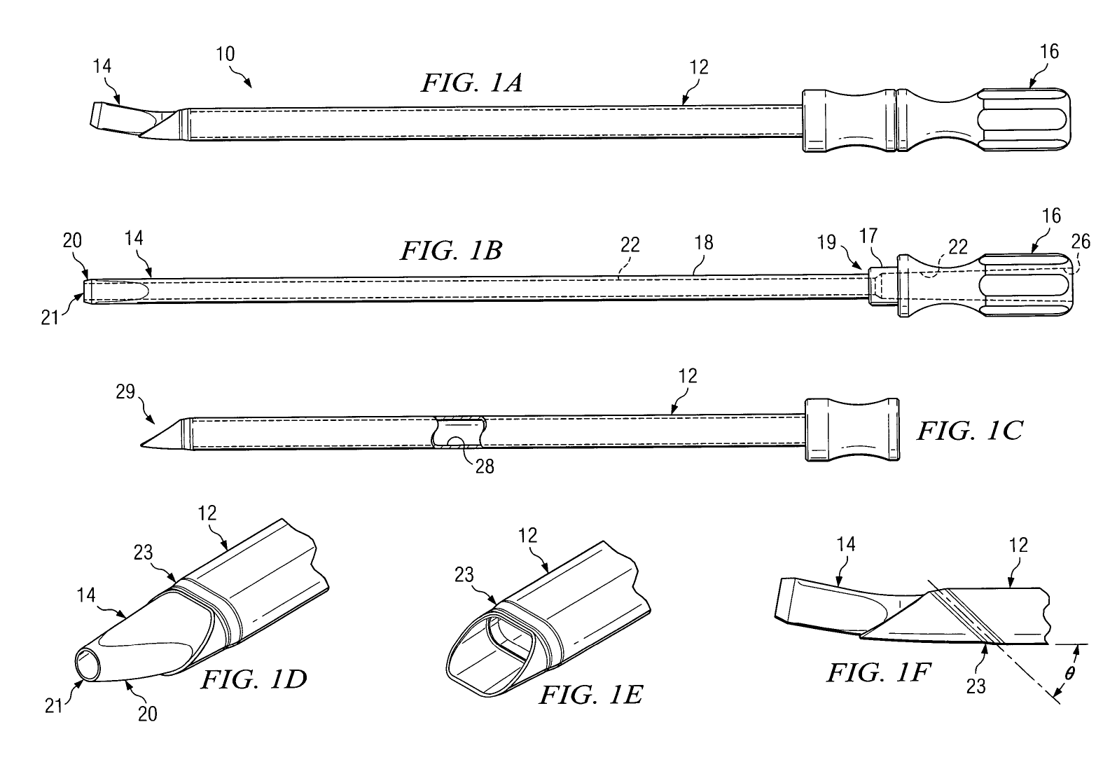

FIG. 1A illustrates an example introducer 10 for implanting a paddle style electrical stimulation lead percutaneously. Introducer 10 may be used to percutaneously introduce a percutaneous or paddle style lead into the epidural space of a user who requires electrical stimulation treatment directed to spinal nerve tissue, for example, for pain management. As known in the art, paddle style leads generally have a width about two times the height of the face of the paddle. The same or an analogous, perhaps smaller, introducer 10 may be used to implant a percutaneous or paddle style lead into other tissue for electrostimulation treatment of a peripheral nerve. In one embodiment, introducer 10 includes an outer sheath 12 and an inner penetrator 14.

FIG. 1B illustrates an example inner penetrator 14 disassembled from outer sheath 12. Inner penetrator 14 includes handle 16, connector 17, and body 18 having proximal end 19 and distal end or tip 20. Tip 20 may be tapered. Connector 17 connects handle 16 to body 18. An inner channel 22 is formed through handle 16 and body 18 and connects opening 26 of handle 16 to opening 21 of tip 20. Inner channel 22 may be configured to attach to a syringe. Inner channel 22 is wide enough to accommodate guide wires of various sizes along which introducer 10 may be advanced during use. Channel 22 may taper or otherwise decrease in diameter as it traverses connector 17 at the handle-body junction. Inner penetrator 14 may be formed from a plastic, such as silastic or another polymer, or any other suitable material. Tip 20 of inner penetrator 14 may be curved as shown in FIGS. 1A-C or may be curved into any other suitable shapes by an operator before inserting the introducer. In certain embodiments, inner penetrator 14 may be bent or curved into a suitable configuration to allow passage around an anatomical obstruction, or formed into any other shape suitable for particular anatomic regions of the body.

FIG. 1C illustrates outer sheath 12 disassembled from inner penetrator 14. The lumen of outer sheath 12 may range in width, for example from approximately 2 mm to approximately 6 mm. The lumen may be oblong, oval, or substantially rectangular as needed to accommodate paddle style leads of various configurations. Outer sheath 12 may taper slightly at tip 29. Tip 29 of outer sheath 12 may be beveled to allow easier passage through tissue and to allow inner penetrator 14 to protrude out of tip 29.

Outer sheath 12 is preferably formed from a metal, such as stainless steel or titanium, or any other suitable material that is stiff and resists bending when outer sheath 12 is inserted through the paravertebral tissue and into the epidural space. In one embodiment, inner penetrator 14 includes tapered tip 20 shown in FIG. 1D. Tapered tip 20 protrudes out of outer sheath 12. Tapered tip 20 preferably allows introducer 10 to pass easily over a guide wire without creating a false passage in an undesirable location in the tissue.

In one embodiment of outer sheath 12, shown in FIGS. 1D-F, tip 20 includes a raised circumferential shoulder or ridge 23 configured to provide an indication or “feel” to a physician as raised ridge 23 comes in contact with the ligamentum flavum. This “feel” occurs when raised ridge 23 comes in contact with the ligamentum flavum causing a slight resistance, pressure, or “notch” feel to the physician as raised ridge 23 comes in contact with and passes through the ligamentum flavum. As many physicians rely on “feel” while performing delicate procedures, this aspect may provide an important indication to the physician as to the location of outer sheath 12 and thus introducer 10 as a whole.

Such a raised ridge 23 can also be applied to needles or cutting devices that otherwise fail to provide physicians sufficient “feel” or a locative indication as the needle cuts through the ligamentum flavum. For example, the edge of outer sheath 12 in FIG. 1E could be configured into a cutting surface for a paddle insertion type needle. The improvement of raised ridge 23 on such a cutting device would provide the needed “feel” or indication to the physician as to where the needle was in the human tissue, thus providing confidence to the physician, as the physician uses such a large needle, that the needle has not yet entered the interthecal space.

Further, raised ridge 23 assists in spreading the fibers of the paravertebral muscle and ligaments as it is inserted. Raised ridge 23 may be angled to assist insertion, for example, at an angle of thirty-five to forty-five degrees or any other angle that would facilitate passage of outer sheath through tissue. During insertion, raised ridge 23 ultimately makes contact with the ligamentum flavum and rests against it during insertion of a guide wire and an electrical stimulation lead.

In one embodiment, outer sheath 12, inner penetrator 14, or both may be formed from radio-opaque material or may include radio-opaque markers that allow the position of outer sheath 12, inner penetrator 14, or both to be visualized with fluoroscopy or plain x-rays, for example, during the insertion process to insure proper positioning in the epidural space.

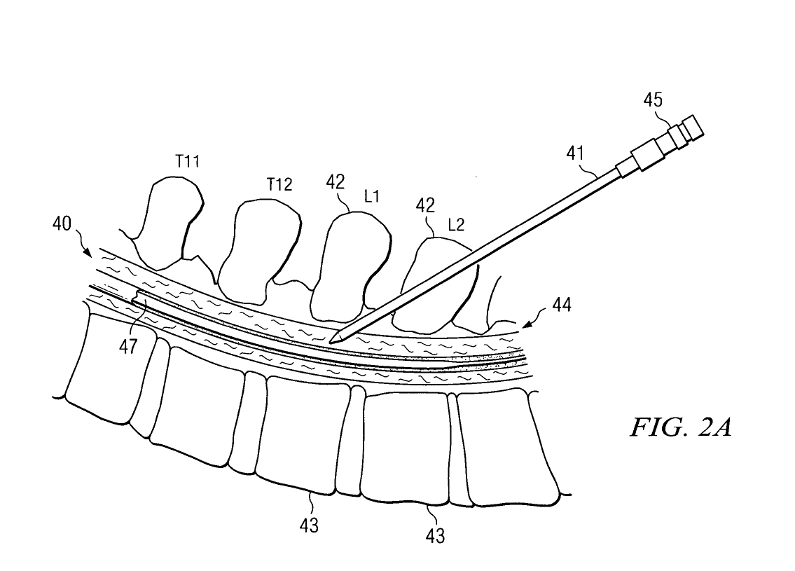

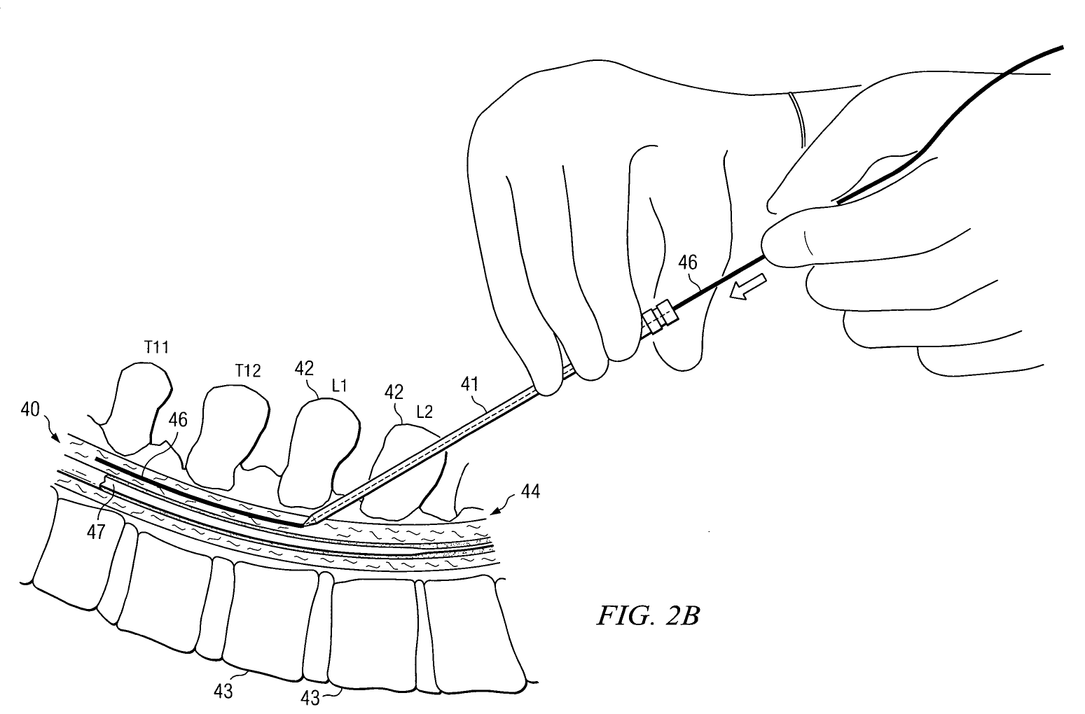

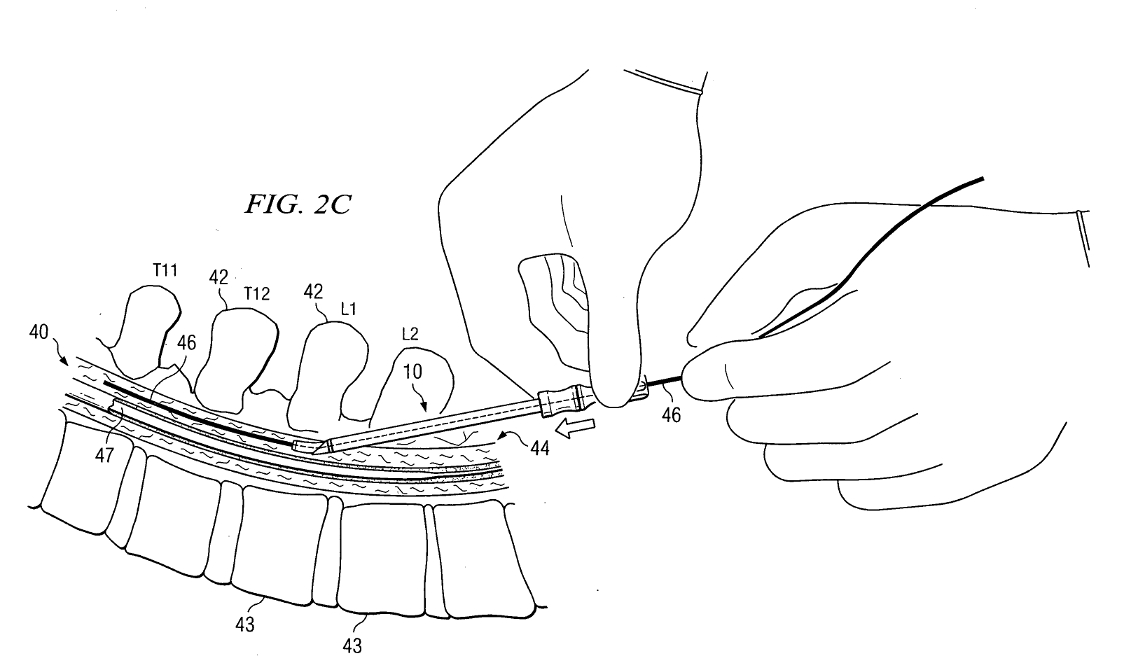

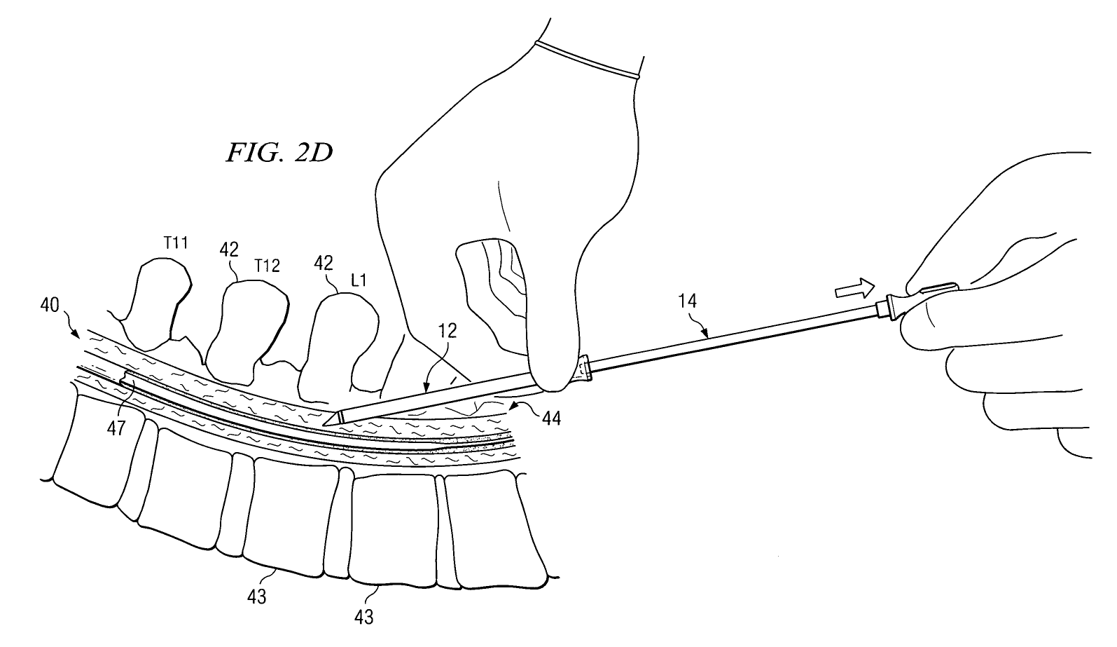

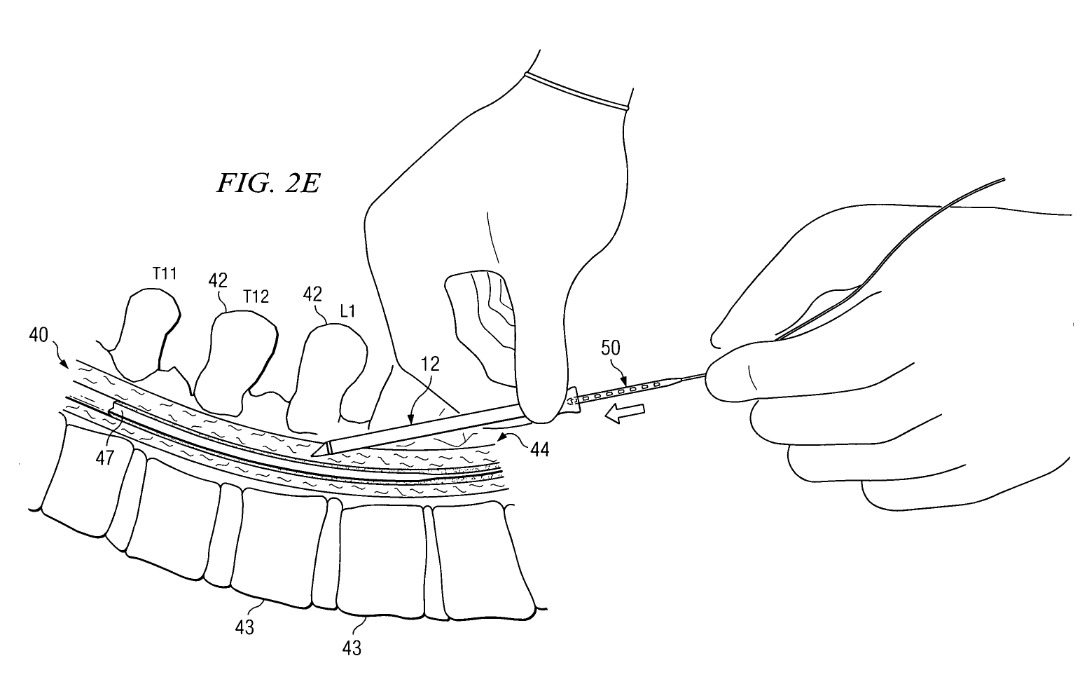

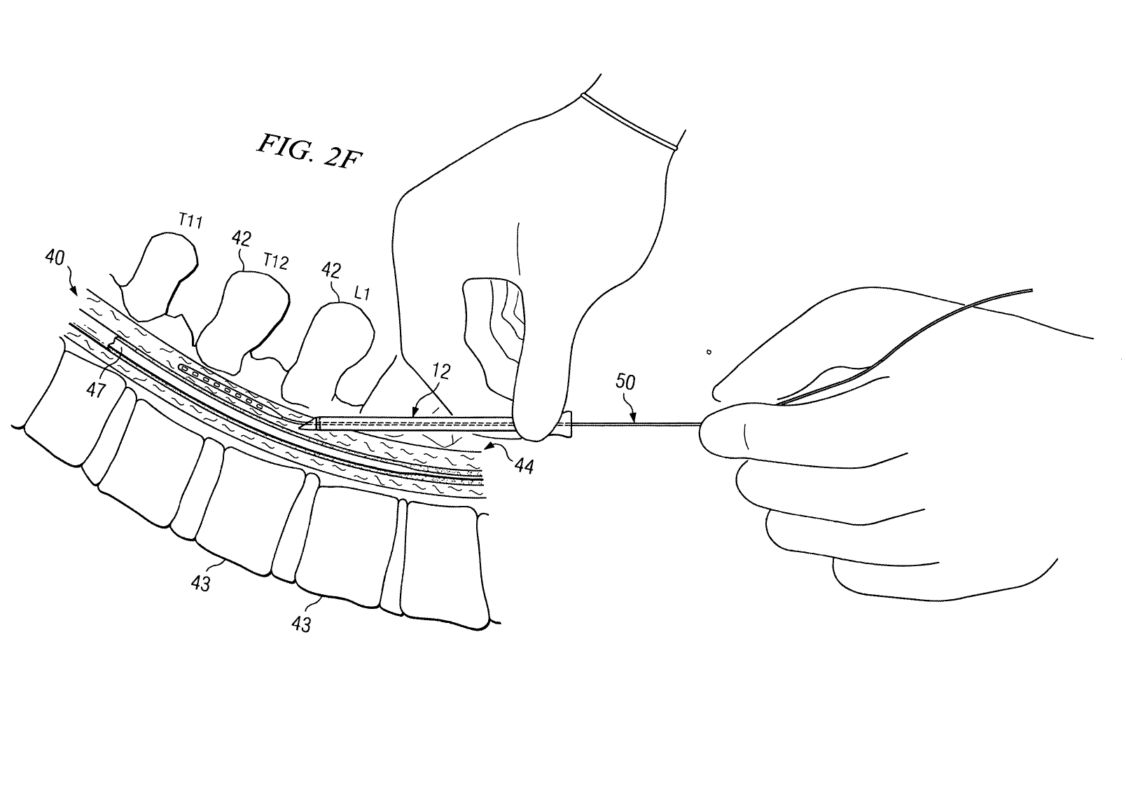

FIGS. 2A-F illustrate an example method of implanting a paddle style electrical stimulation lead into a human's epidural space using an example introducer 10. Spinal cord 47 is also shown. A location between two vertebrae is selected for the procedure. The site may be selected using fluoroscopy. The first step in performing the procedure is to insert needle 41, preferably at an angle, into the skin, and through the subcutaneous tissue and ligamentum flavum 44 of the spine, and into a human's epidural space 40. In one embodiment of the method, for example, the introducer might be inserted at an angle of approximately thirty-five to approximately forty-five degrees. FIG. 2A illustrates insertion of needle 41 through the skin between spinous processes 42 of two vertebrae 43. Entry into epidural space 40 by needle 41 may be confirmed using standard methods such as the “loss-of-resistance” technique after stylet 45, or inner portion of needle 41, is removed.

After removing stylet 45 from needle 41, guide wire 46 may be inserted through needle 41 into epidural space 40, shown in FIG. 2B. A guide wire is used in a preferred embodiment of the method of insertion but is not required to insert a paddle style lead through the introducer. This part of the procedure may be performed under fluoroscopic guidance for example. Fluoroscopy may be used to check the position of guide wire 46 in epidural space 40 before inserting introducer 10. Once the tip of guide wire 46 is within epidural space 40, needle 41 is removed. As shown in FIG. 2C, introducer 10 may then be inserted, preferably at an angle of approximately thirty-five to approximately forty-five degrees, although the exact angle may differ depending on technique and a patient's anatomy, over guide wire 46 and into epidural space 40 using guide wire 46 as a guide. The technique of passing introducer 10 over guide wire 46 helps ensure proper placement of introducer 10 into epidural space 40 and helps avoid inadvertent passage of introducer 10 into an unsuitable location. The operator may choose to cut the skin around the insertion site with a scalpel to facilitate subsequent entry of introducer 10 through the needle entry site.

As introducer 10 is passed through the skin it elongates the hole in the skin made by needle 41. As introducer 10 is passed deeper into the paravertebral tissues, it spreads the fibers of tissue, muscle and ligamentum flavum 44 and forms a tract through these tissues and into epidural space 40, preferably without cutting the tissues. At the level in the tissues where introducer 10 meets and penetrates ligamentum flavum 44 there is a second loss of resistance when inner penetrator 14 has completely penetrated the ligamentum flavum 44. Shoulder or ridge 23 of outer sheath 12 is preferably lodged against ligamentum flavum 44 during insertion of a paddle style lead.

Once introducer 10 has completely penetrated ligamentum flavum, inner penetrator 14 and guide wire 46 may be removed, leaving outer sheath 12 positioned in epidural space 40, as shown in FIG. 2D. As shown in FIG. 2E, paddle style lead 50 may then be inserted through outer sheath 12 and positioned at an optimal vertebral level, using fluoroscopy for example, for the desired therapeutic effect. As shown in FIG. 2F, outer sheath 12 may then be removed leaving only paddle style lead 50 in epidural space 40, where paddle style lead 50 can be further manipulated if necessary to achieve a desired therapeutic effect. Paddle style lead 50 may be secured by suturing it to a spinous process.

As described above, introducer 10 may be used to implant paddle style lead 50 into epidural space 40 for spinal nerve stimulation. The same or an analogous, perhaps smaller, introducer 10 may be used to implant an analogous paddle style lead 50 into any appropriate region of the body for peripheral nerve stimulation. For example, such a paddle style lead 50 may have an outer sheath 12 and lumen 28 with a width of approximately 1 mm to approximately 3 mm.

A similar method of insertion (not expressly shown) may be used to implant a paddle style electrical stimulation lead into a human's peripheral nerve tissue. In this embodiment of the invention a site for insertion in tissue near a nerve is selected. The first step in performing the procedure is to insert a needle into the skin and through the subcutaneous tissue and into tissue near a peripheral nerve. If the needle has a stylet, it may be removed and a guide wire may be inserted through the needle and into the tissue near a peripheral nerve. A guide wire may not be required. Fluoroscopy may or may not be used to guide insertion of a guide wire into tissue near a peripheral nerve. Once the tip of the guide wire, or needle, is in the tissue near a peripheral nerve, introducer 10 may be inserted, preferably at an angle that would depend on the anatomy of the body near the peripheral nerve to be stimulated. As introducer 10 is passed through tissues, it elongates the tract made by a needle or guide wire and spreads the tissue. After positioning introducer 10 in tissue adjacent to the peripheral nerve to be stimulated, inner penetrator 14 is removed. A paddle style lead may then be inserted through outer sheath 12. Outer sheath 12 may then be removed leaving only the paddle style lead in position near the peripheral nerve to be stimulated.

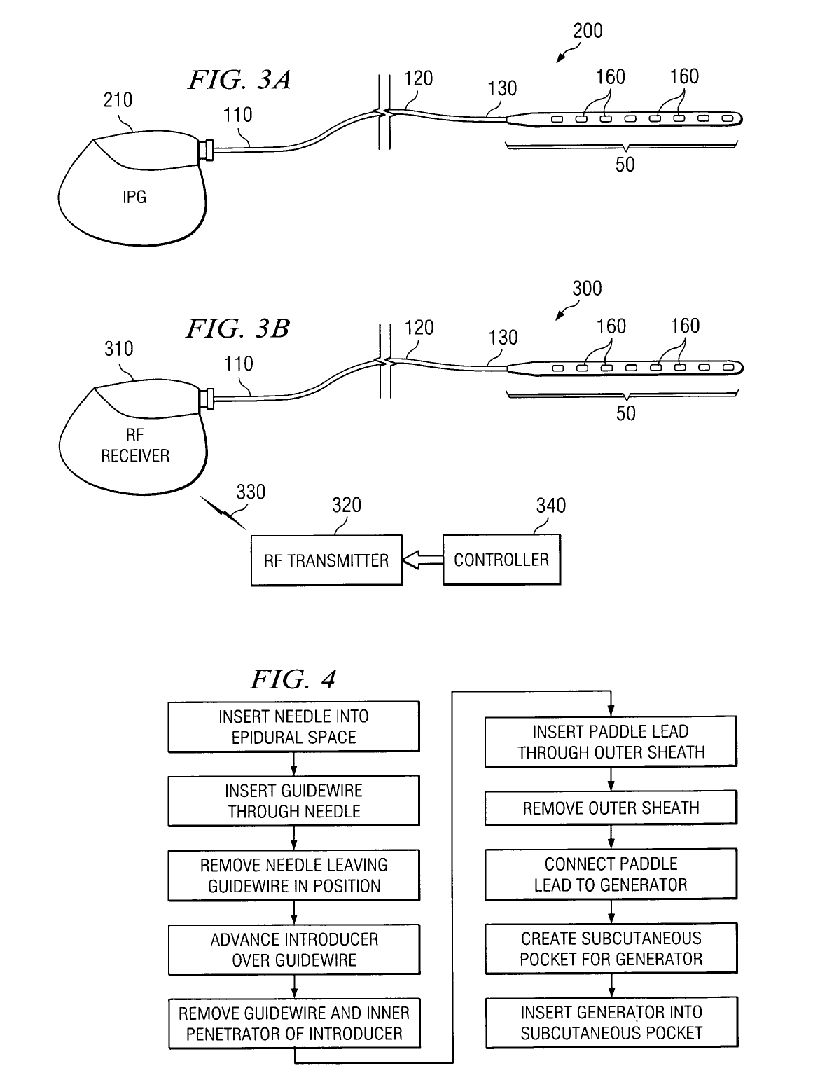

Now referring to FIGS. 3A and 3B, there are shown two embodiments of a stimulation system 200, 300 in accordance with the present invention. The stimulation systems generate and apply a stimulus to a tissue or to a certain location of a body. In general terms, the system 200, 300 includes a stimulation or energy source 210, 310 and a lead 50 for application of the stimulus. The lead 110 shown in FIGS. 3A and 3B is the paddle style lead 50 of the present invention.

As shown in FIG. 3A, the stimulation system 200 includes the lead 50 that is coupled to the stimulation source 210. In one embodiment, the stimulation source 210 includes an implantable pulse generator (IPG). As is known in the art, an implantable pulse generator (IPG) is implanted within the body (not shown) that is to receive electrical stimulation from the stimulation source 210. An example IPG may be one manufactured by Advanced Neuromodulation Systems, Inc., such as the Genesis® System, part numbers 3604, 3608, 3609, and 3644.

As shown in FIG. 3B, the stimulation system 300 includes the lead 50 that is coupled to the stimulation source 310. The stimulation source 310 includes a wireless receiver. As is known in the art, the stimulation source 310 comprising a wireless receiver is implanted within the body (not shown) that is to receive electrical stimulation from the stimulation source 310. An example wireless receiver 310 may be those wireless receivers manufactured by Advanced Neuromodulation Systems, Inc., such as the Renew® System, part numbers 3408 and 3416.

The wireless receiver (not shown) within stimulation source 310 is capable of receiving wireless signals from a wireless transmitter 320. The wireless signals are represented in FIG. 3B by wireless link symbol 330. The wireless transmitter 320 and a controller 340 are located outside of the body that is to receive electrical stimulation from the stimulation source 310. A user of the stimulation source 310 may use the controller 340 to provide control signals for the operation of the stimulation source 310. The controller 340 provides control signals to the wireless transmitter 320. The wireless transmitter 320 transmits the control signals (and power) to the receiver in the stimulation source 310 and the stimulation source 310 uses the control signals to vary the signal parameters of the electrical signals that are transmitted through lead 110 to the stimulation site. An example wireless transmitter 320 may be those transmitters manufactured by Advanced Neuromodulation Systems, Inc., such as the Renew® System, part numbers 3508 and 3516.

As will be appreciated, the connectors are not visible in FIGS. 3A and 3B because the contact electrodes are situated within a receptacle (not shown) of the stimulation source 210, 310. The connectors are in electrical contact with a generator (not shown) of electrical signals within the stimulation source 210, 310. The stimulation source 210, 310 generates and sends electrical signals via the lead 50 to the electrodes 160. Understandably, the electrodes 160 are located at a stimulation site (not shown) within the body that is to receive electrical stimulation from the electrical signals. A stimulation site may be, for example, adjacent to one or more nerves in the central nervous system (e.g., spinal cord) or peripheral nerves. The stimulation source 210, 310 is capable of controlling the electrical signals by varying signal parameters (e.g., intensity, duration, frequency) in response to control signals that are provided to the stimulation source 210, 310.

As described above, once lead 110 is inserted into either the epidural space or near the peripheral nerve, introducer 10 is removed. Lead 110 extends from the insertion site to the implant site (the area of placement of the generator). The implant site is typically a subcutaneous pocket that receives and houses the IPG or receiver (providing stimulation source 210, 310). The implant site is usually positioned a distance away from the stimulation site, such as near the buttocks or other place in the torso area. In most cases, the implant site (and insertion site) is located in the lower back area, and lead 110 may extend through the epidural space (or other space) in the spine to the stimulation site (e.g., middle or upper back, neck, or brain areas). Once the system is implanted, the system of leads and/or extensions may be subject to mechanical forces and movement in response to body movement. FIG. 4 illustrates the steps that may be used to implant a stimulation system 200, 300 into a human.

Although the present invention has been described with several embodiments, a number of changes, substitutions, variations, alterations, and modifications may be suggested to one skilled in the art, and it is intended that the invention encompass all such changes, substitutions, variations, alterations, and modifications as fall within the spirit and scope of the appended claims.

1. An introducer for implanting an electrical stimulation lead to enable electrical stimulation of nerve tissue, comprising:
an outer sheath to accommodate insertion of the electrical stimulation lead through the outer sheath, the outer sheath operable to be inserted into a human body near the nerve tissue; and
an inner penetrator removably housed within the outer sheath and comprising an inner channel configured to accommodate a guide wire, the inner penetrator configured to be advanced along the guide wire to a desired location relative to the nerve tissue and removed from the outer sheath leaving the outer sheath substantially in position for insertion of the electrical stimulation lead through the outer sheath into position proximate the nerve tissue.

2. The introducer of claim 1, wherein the lead is greater than 1.5 millimeters in width.

3. The introducer of claim 1, wherein the electrical stimulation lead has a width of at least approximately two times its height.

4. The introducer of claim 1, wherein the electrical stimulation lead is a paddle style lead.

5. The introducer of claim 1, wherein the nerve tissue comprises spinal nerve tissue and the desired location comprises an epidural space of the human.

6. The introducer of claim 1, wherein the nerve tissue comprises a peripheral nerve.

7. The introducer of claim 1, wherein the inner penetrator comprises a hollow tip configured to extend beyond the outer sheath.

8. The introducer of claim 1, wherein the outer sheath has a raised circumferential ridge configured to create resistance when the circumferential ridge contacts the human's ligamentum flavum.

9. The introducer of claim 1, wherein the inner penetrator and outer sheath comprise one or more radio-opaque markers for visualization using fluoroscopy.

10. The introducer of claim 1, wherein the outer sheath has a substantially oval cross-section.

11. The introducer of claim 1, wherein the outer sheath has a substantially oblong cross-section.

12. The introducer of claim 1, wherein the outer sheath is formed of metal comprising titanium or stainless steel.

13. The introducer of claim 1, wherein the inner penetrator is formed from one or more of plastic, silastic, or a polymeric material.

14. The introducer of claim 1, wherein the tip of the outer penetrator is tapered.

15. The introducer of claim 1, wherein the tip of the inner penetrator and the tip of the outer sheath comprise a curved portion to allow passage of the inner penetrator and outer sheath around an anatomical obstruction.

16. The introducer of claim 1, wherein the inner penetrator is configured to be advanced until an end of the inner penetrator is positioned in the human's epidural space at a desired location relative to the nerve tissue such that the outer sheath forms an insertion tract for the electrical stimulation lead as the inner penetrator advances along the guide wire.

17. A method of implanting an electrical stimulation lead to enable electrical stimulation of nerve tissue, comprising:
inserting a needle into tissue;
removing the needle;
forming a tract for the electrical stimulation lead by spreading tissue using an introducer comprising an outer sheath and inner penetrator removably housed within the outer sheath;
removing the inner penetrator;
leaving the outer sheath substantially in position; and
inserting the electrical stimulation lead through the outer sheath until the electrical stimulation lead is positioned proximate the nerve tissue.

18. The method of claim 17, further comprising:
positioning the guide wire through the needle into a desired location relative to the nerve tissue;
advancing the introducer along the guide wire; and
positioning the introducer at a desired location.

19. The method of claim 17, wherein forming the tract further comprises:
advancing the introducer along a channel created by the needle; and
locating a ligamentum flavum with a circumferential ridge on the outer sheath.

20. The method of claim 17, wherein the electrical stimulation lead comprises a paddle style lead.

21. The method of claim 18, wherein the nerve tissue comprises spinal nerve tissue and the electrical stimulation lead is positioned in an epidural space of a human.

22. The method of claim 17, wherein the nerve tissue comprises a peripheral nerve.

23. The method of claim 18, further comprising verifying the position of one or more of the guide wire, introducer, and the electrical stimulation lead using fluoroscopy.

24. The method of claim 17, wherein the tip of the introducer is tapered and hollow.

25. The method of claim 17, wherein the tip of the introducer comprises a curved portion to allow passage of the introducer around an anatomical obstruction.

26. The method of claim 17, wherein the outer sheath comprises a circumferential ridge configured to create resistance when the circumferential ridge contacts a ligamentum flavum of a human.

27. The method of claim 18, wherein the needle comprises a removable stylet configured to be removed before inserting the guide wire.

28. A method of implanting an electrical stimulation lead in a minimally invasive percutaneous manner to enable electrical stimulation of a human's spinal nerve tissue, comprising:
inserting a needle into the epidural space;
inserting a guide wire through the needle until an end of the guide wire is positioned in the epidural space at a desired location relative to the spinal nerve tissue to be stimulated;
verifying the position of the guide wire in the epidural space using fluoroscopy;
removing the needle and leaving the guide wire substantially in position;
advancing an introducer, comprising an outer sheath and an inner penetrator removably housed within the outer sheath, the inner penetrator of the introducer comprising an inner channel configured to accommodate the guide wire and further comprising a hollow tapered tip configured to extend beyond the outer sheath, the outer sheath of the introducer having a width of at least approximately two times its height, along the guide wire until an end of the inner penetrator of the introducer is positioned in the epidural space at a desired location with respect to the spinal nerve tissue to be stimulated, the outer sheath of the introducer forming a tract as the inner penetrator of the introducer advances along the guide wire;
verifying the position of the introducer in the epidural space using fluoroscopy;
removing the guide wire and the inner penetrator of the introducer and leaving the outer sheath of the introducer substantially in position;
inserting the electrical stimulation lead through the outer sheath of the introducer until the electrical stimulation lead is positioned in the epidural space proximate the spinal nerve tissue to be stimulated; and
verifying positioning of the paddle style electrical stimulation lead in the epidural space using fluoroscopy.

29. A method of implanting a system to enable electrical stimulation of a human's nerve tissue, comprising:
inserting a needle into tissue proximate nerve tissue to be stimulated;
inserting a guide wire through the needle until an end of the guide wire is positioned at a desired location relative to nerve tissue to be stimulated;
removing the needle and leaving the guide wire substantially in position;
advancing along a guide wire an introducer, comprising an outer sheath and an inner penetrator removably housed within the outer sheath, the inner penetrator of the introducer comprising an inner channel configured to accommodate the guide wire and further comprising a hollow tip extending beyond the outer sheath, until an end of the inner penetrator of the introducer is positioned at a desired location relative to nerve tissue to be stimulated, the outer sheath of the introducer forming an insertion tract as the inner penetrator of the introducer advances along the guide wire;
removing the guide wire and the inner penetrator of the introducer and leaving the outer sheath of the introducer substantially in position;
inserting an electrical stimulation lead through the outer sheath of the introducer until the electrical stimulation lead is positioned proximate the nerve tissue to be stimulated;
removing the outer sheath;
connecting the electrical stimulation lead to a generator;
creating a subcutaneous pocket for a generator; and
inserting the generator into the subcutaneous pocket.

30. A system for implanting an electrical stimulation lead to enable electrical stimulation of a human's spinal nerve tissue, comprising:
a needle;
a guide wire;
an introducer comprising an outer sheath and an inner penetrator;
the outer sheath configured to accommodate insertion of the electrical stimulation lead through the outer sheath, the outer sheath operable to be inserted through the human's skin and into the human's epidural space;
an inner penetrator removably housed within the outer sheath and comprising an inner channel configured to accommodate a guide wire;
the inner penetrator configured to be advanced along the guide wire until an end of the inner penetrator is positioned in the epidural space at a desired location relative to spinal nerve tissue to be stimulated such that the outer sheath forms an insertion tract for the electrical stimulation lead as the inner penetrator advances along the guide wire, the inner penetrator configured to be removed from the outer sheath leaving the outer sheath substantially in position in the epidural space for insertion of the electrical stimulation lead through the outer sheath into position proximate the spinal nerve tissue to be stimulated; and
an implantable generator to power the electrical stimulation lead.

31. A needle for introduction of a paddle style electrical stimulation lead near a spinal column of a human, comprising in combination a body having a proximal end and a distal end, a lumen having a continuous oblong cross section defined by a solid outer wall, and a stylet having a handle at a proximal end and a solid body extending from the proximal end to a distal end and adapted to be inserted within the lumen, the improvement comprising:
a raised circumferential ridge configured to create resistance when the circumferential ridge contacts the human's ligamentum flavum.
